(19) United States
(12) Patent Application Publication
(10) Pub. No.: US 2010/0030344 A1
(43) Pub. Date: Feb. 4, 2010

(54) Prosthetic foot with adjustable flat region

(75) Inventors: Andrew H. Hansen, Round Lake, IL (US); Dudley S. Childress, Wilmette, IL (US)

Correspondence Address: Edward J. Timmer, Suite 205, 121 East Front Street, Traverse City, MI, 49684, US

(73) Assignee: Northwestern University

(21) Appl. No.: 12/462,050

(22) Filed: Jul. 28, 2009

Related U.S. Application Data
(60) Provisional application No. 61/137,746, filed on Jul. 31, 2008.

(51) Int. Cl. A61F 2/66

(52) U.S. Cl. 623/53

(57) ABSTRACT

A prosthetic foot device has a foot piece having a forefoot region wherein the forefoot region has one or more adjusting elements for adjusting the length of a flat region of the foot piece for stability in standing.

This application claims benefits and priority of provisional application Ser. No. 61/137,746 filed Jul. 31, 2008, the entire of which is incorporated herein by reference.

### CONTRACTUAL ORIGIN OF THE INVENTION

This invention was made with government support under Contract/Grant No. H133E030030 awarded by the National Institute on Disability and Rehabilitation Research (United States Department of Education) and under Contract/Grant No. R03-HD050428-01 A2 awarded by the National Institute of Health. The government has certain rights in the invention.

### FIELD OF THE INVENTION

The present invention relates to a prosthetic foot device and, more particularly, to a prosthetic foot device with an adjustable length, flat region for stability in standing.

### BACKGROUND OF THE INVENTION

### Clinical Significance

According to data from Adams et al. (1999), the prevalence of the absence of extremities (excluding tips of fingers or toes) was 1,285,000 in the U.S. in 1996. We know from the same sources that approximately 87% of limb amputations are the result of vascular disease. Scandinavian data (Soderberg et al., 2001) indicates that 90% of all lower-limb amputations result from dysvascular conditions, supporting the U.S. data. Meier (1998) has indicated that the majority of lower-limb amputees are more than 50 years of age and that the largest percentage has amputations because of vascular disease that is often associated with diabetes.

Owings and Kozak (1998) indicate that there were 185,000 surgical amputations performed during 1996 in the U.S. (excluding tips of fingers or toes). If finger amputations, toe amputations, and “other” amputations are removed from the data, we are left with 3,000 upper-limb amputations per year and 100,000 lower-limb amputations per year. If 87% of the lower-limb amputations are of vascular origin, about 87,000 amputations per year would be of this variety, leaving around 13,000 amputations per year mostly related to trauma. The limb loss group with vascular disease tends to be older while the group with trauma tends to be younger.

Therefore, in the United States, it is highly likely that most of the prostheses fabricated each year are for K1 and K2 level amputees (see next paragraph for an explanation of the K levels), who may benefit substantially from a more stable ankle-foot prosthetic component. These estimates are based on data that are over ten years old. The older population has increased dramatically in the last ten years, making these estimates conservative.

In 1995, the Medicare Functional Classification Levels (MFCL) were developed to assess the functional abilities of persons with lower-limb amputation (Gailey et al., 2002). The MFCL has 5 level codes: K0, K1, K2, K3, and K4. The lowest level, K0, is for persons who would not benefit from the use of a prosthesis because they do not have the ability or potential to ambulate or transfer. Persons in this level do not receive a prosthesis. Level K1 is for persons who could use a prosthesis to transfer and ambulate on level terrain at a fixed rate. Most K1 level users are limited or unlimited household ambulators. Persons in level K2 are limited community ambulators, capable of minor terrain obstacles, but who cannot significantly vary their cadence. Levels K3 and K4 are reserved for amputees with potential for higher intensity use of their prostheses, with level K4 identifying extremely vigorous persons including athletes, children, and active adults. Unilateral amputees in the higher levels (K3 and K4) may not benefit from the added stability of a bi-modal ankle-foot system because they tend to have good balance and control over their prostheses and a sound limb to assist with balance deficits on the prosthetic side. However, unilateral prosthesis users at the lower functional levels (K1 and K2) and bilateral prosthesis users may have balance and control issues necessitating the use of assistive devices (e.g. canes or walkers). The use of a more stable prosthetic foot may allow them to ambulate without the assistive device or with less reliance on the device, reducing stress to the upper limb controlling the assistive device.

Miller et al. (2001) studied 435 daily users of lower limb prostheses to examine relationships between falling, fear of falling, and balance confidence on mobility and social activity outcomes. Their results indicated that persons who fell in the year prior to the study (fallers) did not score significantly different on their outcome measures than non-fallers. Instead, persons with higher balance confidence scores had significantly higher scores on the mobility and social activity outcomes. The authors suggested that many lower limb prosthesis users expect to fall. In fact, just over half of the persons in the study (52%) reported a fall in the year prior to their participation in the study. The authors postulated that the expectation of falling may diminish the effects of actually falling on mobility and social activity outcomes. They also pointed out that some nonfallers have a fear of falling while some falters do not fear falling (Tinetti et al., 1994 was cited). The authors suggest that training to improve the person's balance confidence could reduce their fear of falling and allow them to be more mobile and socially active. These improvements would likely lead to improved quality of life for lower limb prosthesis users. The appropriate design of the prosthetic ankle-foot system pursuant to the invention can also improve balance confidence in lower limb amputees. If achieved, the increased balance confidence could similarly lead to improved mobility, social activity, and quality of life.

Rockers have been used by many investigators to describe walking. Perry (1992) described the functions of the normal foot and ankle as creating three rockers to facilitate forward progression during walking: the heel, ankle, and forefoot rockers. Morawski and Wojcieszak (1978) studied the use of rockers in walking toys and suggested that rockers could be useful for the design of lower limb prostheses and orthoses. McGeer (1990) created mathematical and physical models of mechanisms that could walk down gentle slopes using only passive dynamic properties (i.e. without the use of external power). A key component of McGeer's model was the circular rocker used to replace the function of the foot and ankle. McGeer (1990) suggested that the “equivalent radius” for human walking would be roughly 0.3 times the length of the leg based on a simple model and calculation. Collins et al. (2005) have developed even more lifelike walking machines that incorporate rockers in place of the feet and ankles, and that are able to walk on level ground. Wisse and van Frankenhuyzen (2003) showed that increasing the radius of the rocker on a passive dynamic walking machine increases the amount of disturbance it can tolerate without falling down, demonstrating a clear relationship between rocker radius and walking stability. Adamczyk et al. (2006) recently examined the effects of wearing rocker boots on metabolic rate of able-bodied ambulators. Subjects were asked to walk at 1.3 m/s on a treadmill while wearing rigidankle walking boots connected to wooden rockers. The metabolic rate was calculated from respiratory gas exchange data measured during treadmill walking trials and was examined as a function of rocker radius. Adamczyk et al. (2006) reported that the subjects walked with a minimum metabolic rate when the rocker radius was approximately 0.3 times the leg length, matching the “equivalent radius” suggested by McGeer (1990). These studies suggest that rockers are important for robust and efficient bipedal ambulation.

Rockers are commonly used on walking casts and walking boots. Hullin and Robb (1991) studied eleven commercially available rockers for application to lower limb casts and found that only two gave walking characteristics that approached those of ablebodied walking. Both of these two attachable rockers were cams, but specific data regarding their radii were not presented. Milgram and Jacobson (1978) described many possible alterations for shoes to treat anomalies of the feet and ankles. A shoe with a constant radius rocker from heel to toe was said to provide an “ankle on the ground”, suggesting that the effect of the ankle could be mimicked by the rocker for walking, eliminating the need for true ankle rotation. It is likely, however, that such a shoe would feel very unstable to its user during tasks that require standing and moderate swaying.

Knox (1996) examined static and dynamic mechanical properties of many prosthetic feet and stated that effective foot shape was key to their function for walking. Knox's work showed that the effective rocker shape of a prosthetic foot, which gradually develops as the foot deforms under the loading conditions of walking, affects the gait of its user. Knox (1996) developed a simple method for measuring the effective rocker shape of the ankle-foot system, and used the method to measure the rocker shapes (referred to later as “roll-over shapes”) of both able-bodied and prosthetic ankle-foot systems. The Shape Foot was developed in applicants' laboratory in the 1990s and consists of a block of wood cut into a rocker shape that is made to attach to a lower limb prosthesis (Knox, 1996). The Shape Foot demonstrated that simple feet could be produced that would have good walking function if an effective rocker shape were used as a main design constraint. However, the Shape Foot was not good for standing.

Further work in applicants' laboratory led to the development of the Shape&Roll prosthetic foot, an inexpensive foot made of copolymer polypropylene/polyethylene that takes a biomimetic shape when loaded during walking (Sam et al., 2004). During development of the Shape&Roll prosthetic foot, questions arose concerning the specific effective rocker shapes that should be used in the design, particularly as amputees encounter different walking conditions in daily life. It was decided to examine the effective rockers used by able-bodied persons during walking and to consider these rockers as the gold standard for development of the Shape&Roll prosthetic foot.

Examinations in applicants' laboratory of able-bodied persons walking under a variety of conditions suggest that persons maintain similar effective rocker shapes during level walking. The effective rocker shape created by the foot and ankle together, the “anklefoot roll-over shape”, appears to maintain the same general form and radius when persons walk at different speeds (Hansen et al., 2004a) and as persons walk with different amounts of weight added to their torso (Hansen, 2002; Hansen and Childress, 2005). The ankle-foot roll-over shape also changes in meaningful ways when women walk with shoes of different heel heights (Hansen and Childress, 2004): When wearing shoes with high heel heights, women adapted to more plantarflexed ankle positions, causing roll-over shapes to be translated downward. The combination of higher heels and increased ankle plantarflexion resulted in orientations of the roll-over shapes that were similar to those achieved when the women walked with lower heeled shoes. The apparent invariance of roll-over shape to level ground walking implies that it could be a useful and simple goal for design of ankle-foot prostheses and orthoses. Able-bodied persons utilize a circular rocker shape for walking on level terrain and maintain this same shape for walking at different speeds (Hansen et al., 2004a), when carrying different amounts of added weight (Hansen & Childress, 2005), or when using footwear of different heel heights (Hansen & Childress, 2004).

Recent studies of prosthesis alignment also support the importance of roll-over shape for level ground walking. Alignment of a prosthesis is the position and orientation of a prosthetic foot with respect to the residual limb socket, and is generally arrived at by a prosthetist using trial-and-error and adjustable hardware in the prosthesis. Our recent study of alignment indicated that experienced prosthetists adjust the alignments of various types of prosthetic feet, each having a different inherent roll-over shape based on mechanical properties, toward a single effective rocker shape with respect to the residual limb socket (Hansen et al., 2003). This finding suggests an “ideal” roll-over shape for walking that prosthetists inadvertently aim to mimic in a person's prosthesis. It seems that this “ideal” shape minimizes gait deviations and patient discomfort, and that is what the prosthetist attempts to find during the dynamic alignment process.

The bulk of previous work on rockers has focused on finding useful shapes for walking. However, for many elderly prosthesis users, standing balance may be equally or even more important.

### SUMMARY OF THE INVENTION

The present invention provides a prosthetic foot device having a foot piece with a forefoot region that has one or more adjusting elements for adjusting the length of a flat region of the foot piece for stability in standing. The length of the flat region can be adjusted as desired for an individual to provide a stable base between a heel region and a forefoot region for stability in standing.

An embodiment of the invention provides a prosthetic foot device that comprises a foot piece having a forefoot region connected to or part of the foot piece. The forefoot region has one or more adjusting elements for controlling the length of the flat region of the foot piece for stability in standing, and yet if desired provide a rocker foot shape for the foot piece in walking. The heel of the prosthetic foot can include a lateral end slot to impart shock absorption to the heel section during walking.

In an illustrative embodiment of the invention, the forefoot region includes a series of transverse adjusting slots that are spaced apart along the length of the forefoot region and that impart flexion thereto. One or more stop adjusting elements are provided in one or more of the tranvserse adjusting slots to control the degree of flexion of the forefoot region in a manner that the length of the flat region of the foot piece for stability in standing can be adjusted to a particular individual user. The one or more stop elements are held in the one or more slots by tight fit therein and/or by a cosmetic or other cover on the prosthetic foot device.

The present invention provides a prosthetic foot device that offers an adjustable flat foot region for stability for standing and yet permits a rocker foot shape for walking. Providing an individually adjustable flat foot region for standing establishes an inherently stable base for individuals and may reduce the occurrence of falls. This feature is quite advantageous since the majority of lower limb prosthesis users in industrialized nations are in the lowest functional levels and many had their amputations as a result of diabetes or vascular disease, Many of these users are older and have balance issues. Loss of sensation due to their systemic disease is also common. Falling is common in this group of prosthesis users. Providing a flat region in standing provides a stable base for these individuals and may reduce the occurrence of falls.

Other advantages and benefits of the present invention will become more readily apparent from the following detailed description taken with the following drawings.

### BRIEF DESCRIPTION OF THE DRAWINGS

FIG. 1 is a schematic elevation of a prosthetic foot device in accordance with an embodiment of the invention.

FIG. 2 is a schematic plan view of the prosthetic foot device of FIG. 1.

FIG. 3 is a schematic elevation of a prosthetic foot device in accordance with an embodiment of the invention with no stop elements residing the transvese adjusting slots and having a rocker foot shape during walking.

FIG. 4 is a schematic elevation of a prosthetic foot device in accordance with an embodiment of the invention with one stop element residing in one transvese adjusting slot.

FIG. 5 is a schematic elevation of a prosthetic foot device in accordance with an embodiment of the invention with two stop elements residing respective transvese adjusting slots.

FIGS. 6A, 6B, 6C, and 6D show effective foot shapes during walking (FIG. 6A), during quiet standing (FIG. 6B), during low amplitude swaying (FIG. 6C), and during higher amplitude swaying (including going up the toes and heels (FIG. 6D).

### DETAILED DESCRIPTION OF THE INVENTION

The present invention provides a prosthetic foot device having a foot piece with a forefoot region that includes one or more adjusting elements for adjusting the length of a flat region of the foot piece for stability in standing. The length of the flat region can be adjusted as desired for an individual to provide a stable base between a heel region and a forefoot region for stability in standing.

The present invention embodies observations of a 25 year old able-bodied female subject and others who participated in a pilot study to indicate the effective rockers used during walking, standing, and swaying. A modified Helen Hayes marker set (Kadaba et al., 1990) was placed on the subject. For each of the tasks, the subject's center of pressure of the ground reaction force was transformed from a laboratory-based coordinate system to a body-based coordinate system. The body-based coordinate system was created in the sagittal plane using the ankle marker as the origin. The y-axis of the body-based coordinate system went from the ankle and through a virtual hip marker (sagittal projections of these markers). The x-axis went through the ankle, was perpendicular to the y-axis, and also remained in the sagittal plane. This method has been used by applicants to indicate the effective rocker, or rollover shape, that the physiologic knee-ankle-foot system conforms to during walking (Hansen et al., 2004a; Hansen et al., 2004b; Hansen and Childress, 2004; Hansen and Childress, 2005).

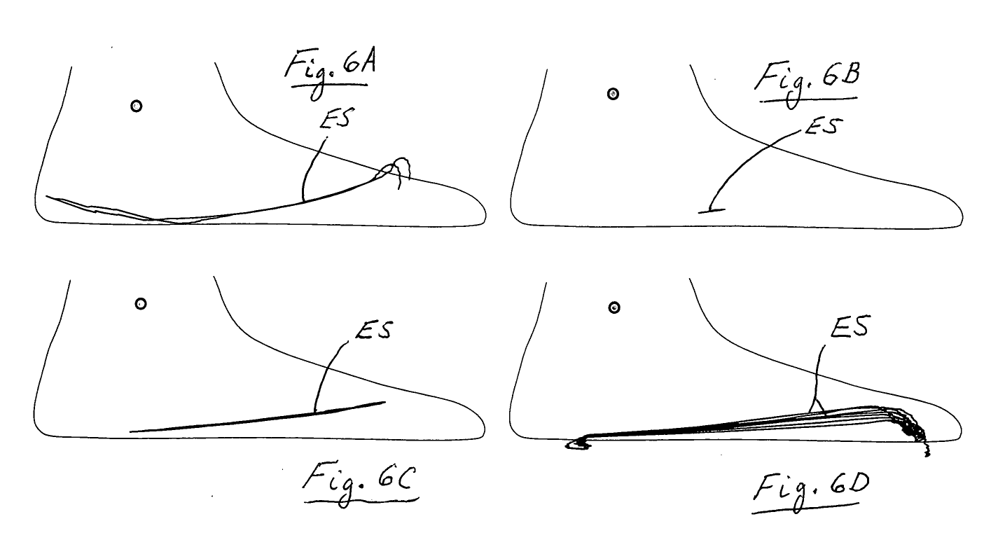

The female subject was asked to walk at her freely-selected walking speed while kinematic and kinetic data were collected. After the walking trials, the subject was asked to stand quietly for at least 10 seconds while data were collected. The subject was also asked to do small amplitude swaying in the anterior-posterior direction as well as large amplitude swaying (that required her to go up on her toes and heels) for at least 10 seconds per trial. The effective rockers that were calculated are shown in FIG. 6A through 6D. The circles indicate the ankle marker, which is the origin of the leg-based coordinate system. Foot outlines are drawn for reference purposes and are not necessarily to scale.

The effective rocker shapes ES that were calculated are shown in FIGS. 6A through 6D. The walking shapes (FIG. 6A) are curved and look similar to knee-ankle-foot roll-over shapes previously reported for able-bodied ambulators (Hansen et al., 2004a). The effective rocker measured during quiet standing is short and appears to be flat (FIG. 6B). When the subject did small amplitude swaying, a longer effective shape was seen that was also very flat (FIG. 6C). Finally, when the subject performed large amplitude swaying in which she went up on the heels and toes, the effective rocker is flat with downward dipping ends (see FIG. 6D). The effective rocker shapes shown for this subject indicate that the radii of these rocker shapes should be different for walking and standing. Creating a circular arc for walking and a flat shape for standing may be biomimetic. The invention described here refers to a compromise shape for standing and walking (a curved shape with a flat region) that can be adjusted to suit the needs of each individual amputee.

Further studies of the effective shapes of the ankle-foot system during walking, standing, and swaying have just been completed in the applicants' laboratory with similar results. The applicants measured effective ankle-foot rocker shapes used by eleven able-bodied persons during walking, swaying, and standing. The radius (measured as the inverse of the average curvature for the shape) was found to be about ⅓ of the leg length for walking, but over two times the leg length for swaying. The difference in curvature between walking and swaying shapes was highly significant (p=0.003).

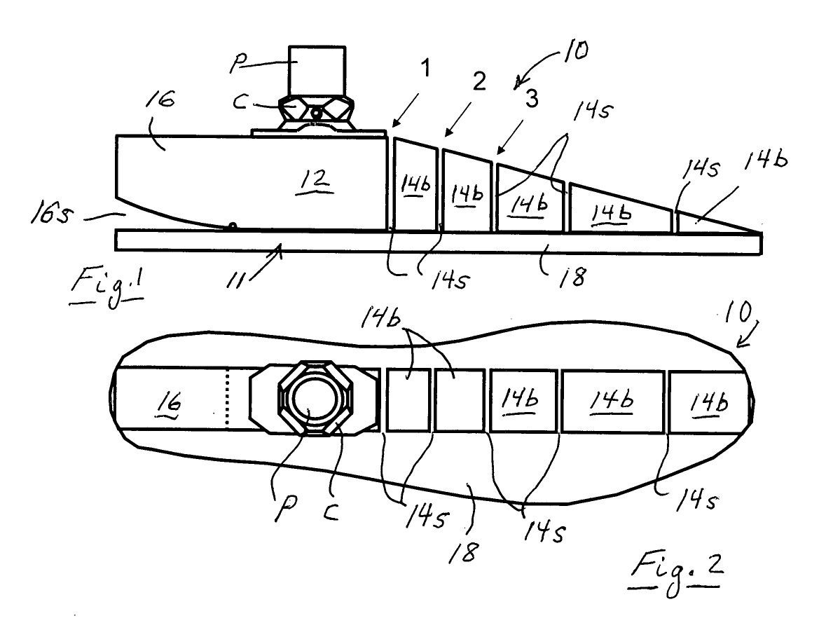

Referring to FIGS. 1 and 2, a prosthetic foot device 10 is shown for providing an effective foot rocker shape that mimics that of an able-bodied ankle-foot system during walking [as described by Sam, M., Childress, D. S., Hansen, A. H., Meier, M. R., Lambla, S., Grahn, E. C., Rolock, J. S (2004). The Shape&Roll prosthetic foot (Part 1): Design and development of appropriate technology for low-income countries. Med Confl Surviv 20(4), 294-306]. This foot device is biomimetic, and yet it is light in weight and it can be manufactured at a very low cost.

In accordance with an embodiment of the invention shown in FIGS. 1 and 2, the prosthetic foot device 10 comprises a foot piece 11 comprising a central block region 12 disposed under the connection location C of the prosthetic foot device with to the pylon P and residual limb socket (not shown), an elongated forefoot region 14, and a heel region 16 residing on an elongated flexible sole plate 18 defining the length of the foot device. The central block region 12, forefoot region 14, heel region 16 and sole plate 18 can be separate pieces connected together or they can be made a one-piece unitary component by, for example, a one-piece plastic injection molding or casting.

In accordance with an illustrative embodiment of the invention, the forefoot region 14 includes one or more adjusting elements shown as slots 14s for adjusting the length L of a flat region R of the foot piece 111 for stability in standing, see FIGS. 3, 4, and 5 showing flat region R with different adjusted lengths L. The length L of the flat region R can adjusted as desired for an individual to provide a stable base between a heel region and a forefoot region for stability in standing. The flexible forefoot region R also provides a rocker foot shape for walking as illustrated in FIGS. 3, 4, and 5.

Referring to FIGS. 1 and 2, the forefoot region 14 includes a series of transverse adjusting slots 14s between block regions 14b that are spaced apart along the length of the forefoot region and that impart flexion thereto. The adjusting slots 14s extend transverse (e.g. perpendicular) to the length of the forefoot 14. One or more stop adjusting elements 22 are provided in one or more of the tranvserse adjusting slots 14s to control the degree of flexion of the forefoot region 14 in a manner that the length L of the flat region R of the foot piece 111 for stability in standing can be adjusted to a particular individual user, FIGS. 3, 4, and 5.

The stop elements 22 can comprise rigid stop elements made of hard plastic or any other hard material and formed by molding, casting, machining, and other technique. The main requirement of the rigid stop elements 22 is that each stop element fill the respective individual slot 14s tightly. If the thickness of the individual slot 14s (cut) is 1 mm, for example, the rigid stop element 22 should be 1 mm or slightly over to provide a tight fit. These stop elements 22 can be slid into the respective individual slot 14s from the top and fill the entire slot, or each stop element can slide in only partially into each slot and have a rim around the top that prevents the stop element going into the slot 14s too deeply. The latter configuration would allow use of similar size rigid stop elements for all of the slots 14s (with a maximum height equal to the depth of the shortest slot).

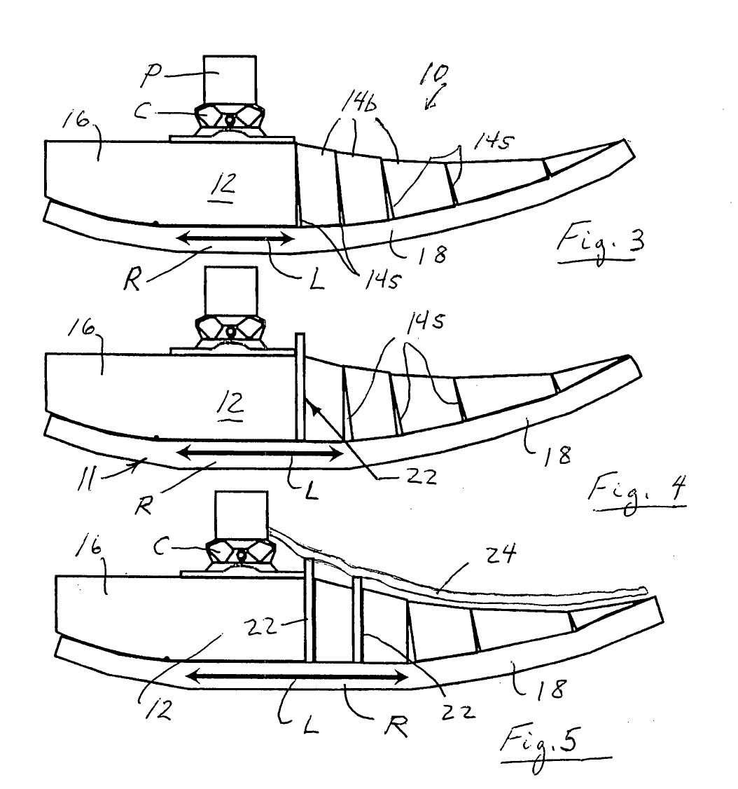

The one or more stop adjusting elements 22 can be held in the respective individual one or more slots 14s by their tight fit (friction fit) and the fact that during walking or standing there is increased pressure on them that tends to keep them from slipping out of the slots 14s. If the prosthetic foot device has a cosmetic shell or cover 24 (partially shown in FIG. 5) placed thereon, the rigid stop elements 22 residing in the slots can be held in place by the cover. That is, the cover itself would prevent the stop elements 22 from falling out of the slots 14s.

Each slot 14s essentially creates a flexural hinge of the forefoot region 14 with a limited range of motion. As the person rolls over during walking, the appropriate effective rocker shape is provided by the closure of these forefoot region slots 14s. The placement of the slots, slot width, and the forefoot profile all factor into the effective rocker shape that is attained when walking with the foot. The appropriate slot spacing can be determined depending on the slot width, the forefoot profile, and the preferred radius of the rocker shape for walking.

Referring to FIG. 1, the heel region 16 may include a lateral end slot 16s whose closure during walking provides a shock absorbing effect in the early stance phase of walking. The end slot 16s diverges as it extends from the central block region 12 to the outermost end of the heel region 16 to form a wedge slot or cut therein.

In manufacture of a prototype of the prosthetic foot device described above, applicants formed the adjusting slots 14s and end slot 16s by making cuts with a saw blade in a unitary plastic block to yield the separated forefoot blocks 14b and slots 14s shown. The heel slot 16s similarly was made using saw blade to cut the slot in the same plastic block. Hence, the slots 14s and 16s may be referred to herein as cuts. Those skilled in the art will appreciate that slots 14s and 16s are not limited to those formed by saw cuts and can be formed in any suitable manner including molding, casting, machining, and any other technique.

Between the heel slot 16s and the first forefoot slot 14s, there is the flat (“flat spot”) region R, which in an illustrative embodiment is approximately 45 mm in length L. This “flat spot” region is located under the connection location C of the foot piece 11 with the remainder of the prosthesis and essentially connects the flexural components of the foot piece (the sole plate 18 with forefoot blocks 14b) to the remainder of the prosthesis (i.e. the pylon P and residual limb socket). Applicants experimented with different lengths of this “flat spot” region R by altering the length of the wedge slot 16s in the heel region R and have found that a threshold length of this “flat spot” region is needed to prevent tensile failure at the proximal end of the heel slot 16s during forefoot loading. This “flat spot” length can be increased in the anterior direction by physically blocking different numbers of forefoot slots 14s (see FIGS. 3, 4, and 5). The first four slots (cuts) 14s of the forefoot region are spaced at intervals of approximately 6% of the foot's length. Because of this spacing, slots 14s numbered 1, 2, and 3 lead to increases in length of the “flat spot” region R of approximately 6%, 12%, and 18% of foot length, respectively. A finer resolution in control of the length of the “flat spot” region R is possible without changing the overall roll-over shape by placing more slots 14s in the forefoot region 14 and using different slot blocking schemes.

The addition or substraction of stop elements 22 to the slots 14s of the forefoot region will give the amputee the flexibility to choose an appropriate “flat spot” region during the setup of the prosthetic foot device and will allow the amputee to easily adjust the required stability of the prosthetic foot device over time. Additionally, this adjustability of the stability of the foot device can be useful in the training of new amputees to stand and then walk. For example, the new amputee could start out with a very stable foot on their prosthesis using several stop elements 22 in several forefoot slots 14s while learning to stand and can slowly be given more flexibility in the forefoot region 14 by the simple removal of stop elements 22 from some of the forefoot slots. This method of training may give new amputees more confidence while they are initially learning to use the prosthesis.

The present invention provides a prosthetic foot device that offers an adjustable flat foot region for stability for standing and yet permits a rocker foot shape for walking. Providing an individually adjustable flat foot region for standing establishes an inherently stable base for individuals and may reduce the occurrence of falls. This is quite advantageous since the majority of lower limb prosthesis users in industrialized nations are in the lowest functional levels and many had their amputations as a result of diabetes or vascular disease. Many of these users are older and have balance issues. Loss of sensation due to their systemic disease is also common. Falling is common in this group of prosthesis users. Providing a flat region in standing provides a stable base for these individuals and may reduce the occurrence of falls. The prosthetic foot device foot should be simple and inexpensive to fabricate and may be modified such that it should be easy to manufacture using automated processes including injection molding or compression molding. There may be other commercial applications for foot devices with adjustable flat regions, for example in the manufacture of walking robots.

Although the invention has been described with respect to certain embodiments for purposes of illustration, those skilled in the art will appreciate that changes and modifications can be made therein within the scope of the invention as set forth in the appended claims.

### REFERENCES

### Which are Incorporated Herein by Reference

Adamczyk, P. G., Collins, S H. and Kuo, A. D. (2006). The advantages of a rolling foot in human walking. J Exp Blot 209, 3953-3963.
Adams P F, Hendershot G E, and Marano M A. (1999). Current estimates from the National Health Interview Survey, 1996. National Center for Health Statistics. Vital Health Stat 10(200).
Collins, S., Ruina, A., Tedrake, R., Wisse, M. (2005). Efficient bipedal robots based on passive-dynamic walkers. Science 307, 1082-1085.
Galley, R. S., Roach, K. E., Applegate, E. B., Cho, B., Cunniffe, B., Lict, S., Maguire, M, and Nash, M. S_ (2002). The amputee mobility predictor: an instrument to assess determinants of the lower-limb amputee's ability to ambulate. Arch Phys Med Rehabil 83, 613-627.
Gard, S. A, and Childress, D. S (2001). What determines the vertical displacement of the body during normal walking? J Prosthet Orthot 13, 64-67.
Hansen, A. H. (2002). Roll-over characteristics of human walking with applications for artificial limbs. Ph.D. thesis, Evanston, Northwestern University
Hansen, A. H., Meier, M. R., Sam, M., Childress, D. S. and Edwards, M. L. (2003). Alignment of trans-tibial prostheses based on roll-over shape principles. Prosthet Orthot Int 27(2), 89-99.
Hansen, A. H, Childress, D. S., and Knox, E. H. (2004a). Roll-over shapes of human locomotor systems: effects of walking speed. Clin Biomech 19(4), 407-14.
Hansen, A., H., Childress, D. S., Miff, S. C. (2004b) Roll-over characteristics of human walking on inclined surfaces. Hum Movement Sci. 23(6), 807-821.
Hansen, A., Sam, M., Childress, D. (2004c) The Effective Foot Length Ratio (EFLR) A Potential Tool for Characterization and Evaluation of Prosthetic Feet. J Prosthet Orthot, 16(2), 41-45.
Hansen, A. H. and Childress, D. S. (2004). Effects of shoe heel height on biologic rollover characteristics during walking. J Rehabil Res Dev 41(4), 547-554.
Hansen, A. H. and Childress, D. S. (2005) Effects of Adding Weight to the Torso on Roll-over Characteristics of Walking. J Rehabil Res Dev, 42(3), 381-390.
Hullin, M. G. and Robb, J. E. (1991). Biomechanical effects of rockers on walking in a plaster cast. J Bone Joint Surg Br 73(1), 92-5. Jhoun, J. (1997), Studies of human standing, stepping, and gait initiation. Ph.D. thesis, Evanston, Northwestern University.
Kadaba, M. P., Ramakrishnan, H. K. and Wootten, M E. (1990). Measurement of lower extremity kinematics during level walking. J Orthop Res 8(3), 383-92.
Knox, E. H. (1996). The role of prosthetic feet in walking. Ph.D. thesis, Evanston, Northwestern University.
McGeer, T. (1990). Passive dynamic walking. Int J Robot Res 9(2), 62-82.
Meier, R. (1998) High Tech and the Active Older Amputee. Conference Book of the 9th World Congress of the International Society of Prosthetics and Orthotics, Amsterdam, The Netherlands
Miff S C, Hansen A H, Childress D S, Gard S A, Meier M R. Roll-over Shapes of the AbleBodied Knee-Ankle-Foot System during Gait Initiation, Steady-State Walking, and Gait Termination. Gait and Posture, in press.
Milgram, J. E. and Jacobson, M. A. (1978). Footgear: therapeutic modifications of sole and heel. Orthopaedic Review 7(11), 57-62.
Miller, W. C., Deathe, B., Speechley, M., and Koval, J. (2001). The influence of falling, fear of falling, and balance confidence on prosthetic mobility and social activity among individuals with a lower extremity amputation. Arch Phys Med Rehabil 82, 1238-1244.
Morawski, J. and Wojcieszak, I. (1978). Miniwalker-a resonant model of human locomotion. Biomechanics VIA. E. Asmussen, Jorgensen, K. Baltimore, University Park Press. 2A: 445-451.
Nashner, L. M. (2001). Computerized dynamic posturography. Practical Management of the Dizzy Patient Goebel, J. A., Philadelphia, Lippincot Williams & Wilkins. 143-170.
O'Loughlin, J. L., Robitaille, Y., Boivin, J., and Suissa S. (1993). Incidence of and risk factors for falls and injurious falls among the community-dwelling elderly. Am J Epidemiol 137, 342-354.
Owings M F, Kozak L J. (1998). Ambulatory and inpatient procedures in the United States, 1996. National Center for Health Statistics. Vital Health Stat 13(139).
Perry, J. (1992). Gait Analysis: Normal and Pathological Function. Thorofare, SLACK Incorporated.
Sam, M., Childress, D. S., Hansen, A. H., Meier, M. R., Lambla, S., Grahn, E. C., Rolock, J. S (2004). The Shape&Roll prosthetic foot (Part 1): Design and development of appropriate technology for low-income countries. Med Confl Surviv 20(4), 294-306.
Sattin, R. W. (1992). Falls among older persons: A public health perspective. Annu Rev Pubi Health 13, 489-508.
Soderberg, B., Wykman, A., Schaarschuch, R., Persson, B. M. (2001) Partial Foot Amputations: Guidelines to Prosthetic and Surgical Techniques. Second Edition. Swedish Orthopaedic Association's Publication Series No. 13. Helsingborg, Sweden.
Tinetti, M. E., Speechley, M., and Ginter, S. F. (1988). Risk factors for falls among elderly persons living in the community. N Engi J Med 319, 1701-1707.
Tinetti, M. E., and Speechley, M. (1989). Prevention of falls among the elderly. N Engi JMed 320, 1055-1059.
Tinetti, M. E., Mendes de Leon, C. F., Doucette, J. T., and Baker, D. I. (1994). Fear of falling and fall-related efficacy in relationship to functioning among community-dwelling elders. J Gerontol 49, M140-147.
Wiebusch, C. F. (1939) The Spring Clutch. Journal of Applied Mechanics, September, A1 03-A1 08.
Wisse, M. and van Frankenhuyzen, J. (2003). Design and Construction of MIKE; a 2D autonomous biped based on passive dynamic walking. Proceedings of the AMAM Conference Of Adaptive Motion of Animals and Machines, Kyoto, Japan.

We claim:

1. A prosthetic foot device, comprising a foot piece having a forefoot region wherein the forefoot region has one or more adjusting elements for adjusting the length of a flat region of the foot piece for stability in standing.

2. The foot device of claim 1 wherein the forefoot includes a series of transverse adjusting slots along its length.

3. The foot device of claim 2 including one or more stop elements residing in one or more of the tranvserse slots.

4. The foot device of claim 3 wherein the one or more stop elements are held in the one or more transverse slots by tight-fit therein and/or by a cover on the prosthetic foot.

5. The foot device of claim 1 including a heel region having a lateral end slot.

6. The foot device of claim 1 wherein the foot piece further comprises a flexible sole plate under the forefoot region.

7. A method of adjusting a length of a flat region of a prosthetic foot for stability in standing, comprising limiting the range of flexion of a forefoot region of the prosthetic foot by one or more adjusting elements in a manner to control the length of the flat region to provide stability in standing for an individual.

8. The method of claim 7 including placing one or more stop elements in one or more tranvserse adjusting slots in the forefoot region of the foot piece of the device.

9. The method of claim 7 including slotting a heel region to impart flexibility to the heel region.

10. The method of claim 6 including holding the one or more stop elements in one or more transverse slots in the forefoot region using tight-fit and/or a cover of the prosthetic foot.
